(19) United States
(12) Patent Application Publication
(10) Pub. No.: US 2018/0154402 A1
(43) Pub. Date: Jun. 7, 2018

(54) Steam Cleaner for Baby Products

(71) Applicant: Taylor Beyl, Keystone, CO (US)

(72) Inventor: Taylor Beyl, Keystone, CO (US)

(21) Appl. No.: 15/367,614

(22) Filed: Dec. 2, 2016

(51) Int. Cl. B08B 3/02

(52) U.S. Cl. CPC B08B 3/02; B08B 2230/01

(57) ABSTRACT

The current invention is a steam cleaner that is oval in shape. The cleaner has a removable lid attached to a base. The steam cleaner has a digital timer that illuminates green yellow and red. The steam cleaner will use dual chambers. The base will have exchangeable guides that will hold the bottles and other baby products in place in the base for cleaning. The steam cleaner will have stabilizing rings and platforms to assist in the placement and cleaning of the baby products.

### BACKGROUND

### 1. Field of the Invention

The invention relates generally to a steam cleaning device, and in particular to one to be used with baby products.

### 2. Description of Prior Art

There are currently a large number of devices current being uses to clean baby products. Some of the current products range from simple bags, which allow water to be boiled in a microwave sanitizing the baby product in the same bag to large machines that sit on a countertop and steam clean or sanitize baby products over a period of time.

There is still room for improvement in the art.

### SUMMARY OF THE INVENTION

A current invention is a steam cleaner for baby products.

The steam cleaner is oval in shape with a removable lid attached to a base. The steam cleaner has a digital timer that illuminates green yellow and red. The steam cleaner will use dual chambers. The base will have a guide that will hold the bottles and other baby products in place in the base for cleaning. The steam cleaner will have stabilizing rings and platforms to assist in the placement and cleaning of the baby products.

The steam cleaner can clean up to six bottles as well as nipples.

The current invention is more efficient, effective and functional than the current art.

### BRIEF DESCRIPTION OF THE DRAWINGS

Without restricting the full scope of this invention, the preferred form of this invention is illustrated in the following drawings:

FIG. 1 shows a view of the steam cleaning device;

FIG. 2 shows a view of the steam cleaning device with the bottles facing down;

FIG. 3 shows a view of the steam cleaning device with the lid open;

FIG. 4 shows another view of the steam cleaning device with the lid open;

FIG. 5 shows a top view of the steam cleaning device with the lid open;

FIG. 6 shows another top view of the steam cleaning device with the lid open;

FIG. 7 shows another top view of the steam cleaning device with the lid open;

FIG. 8 shows a front top perspective view; and

FIG. 9 shows a bottom view of the cleaning device.

### DESCRIPTION OF THE PREFERRED EMBODIMENT

There are a number of significant design features and improvements incorporated within the invention.

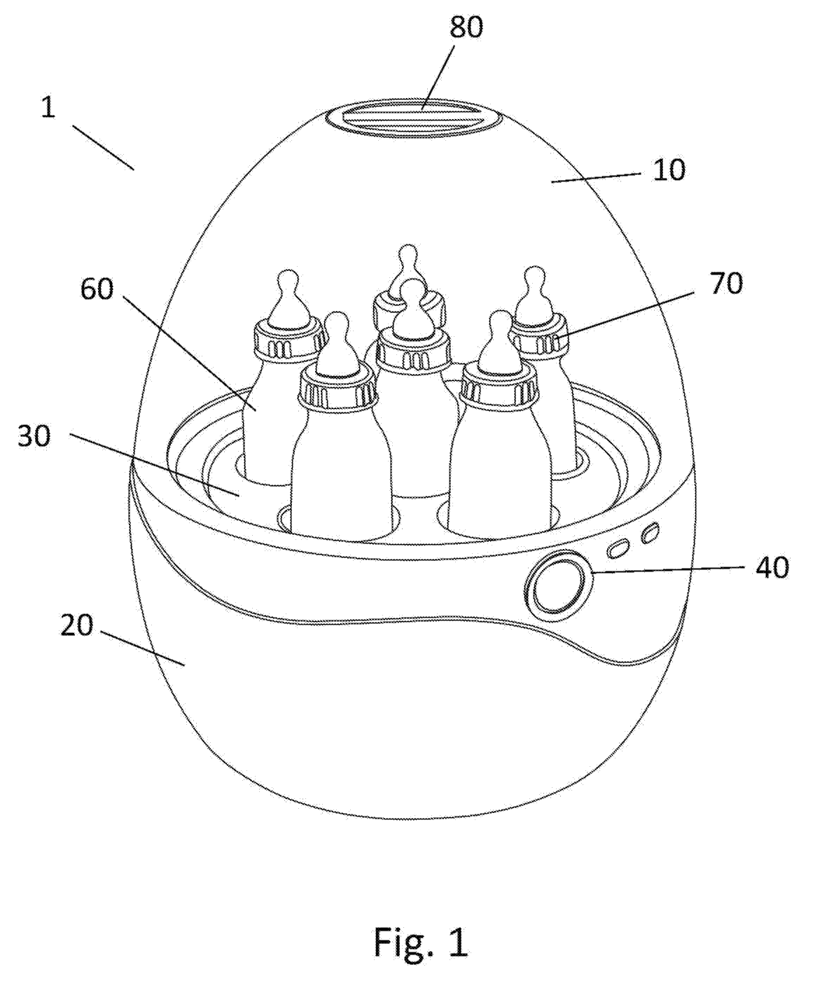

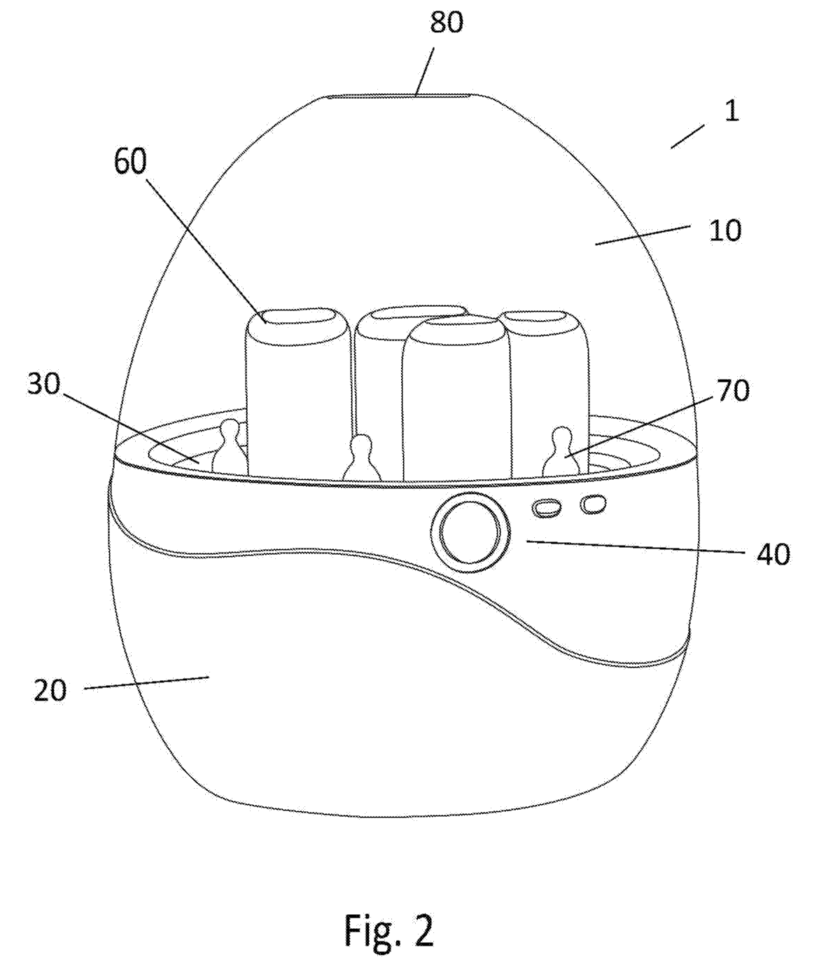

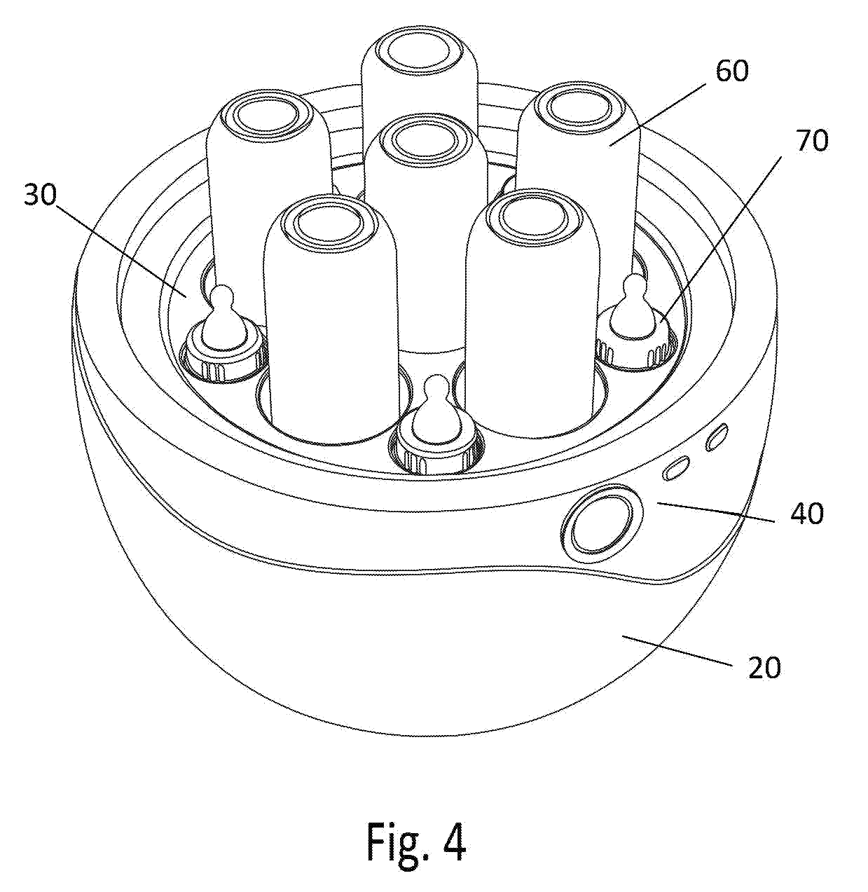

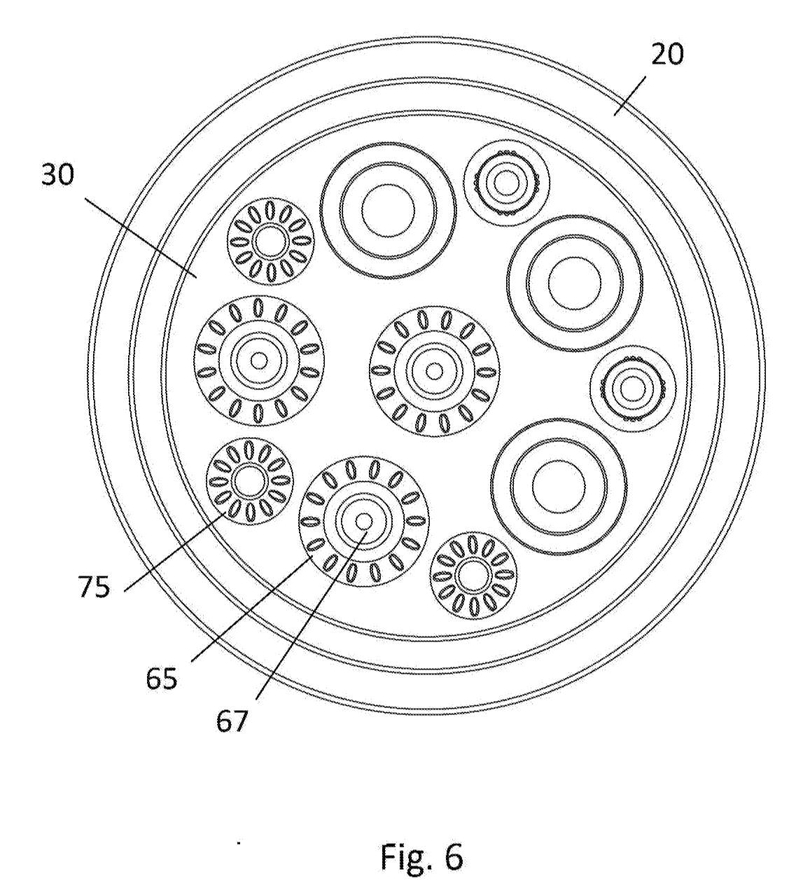

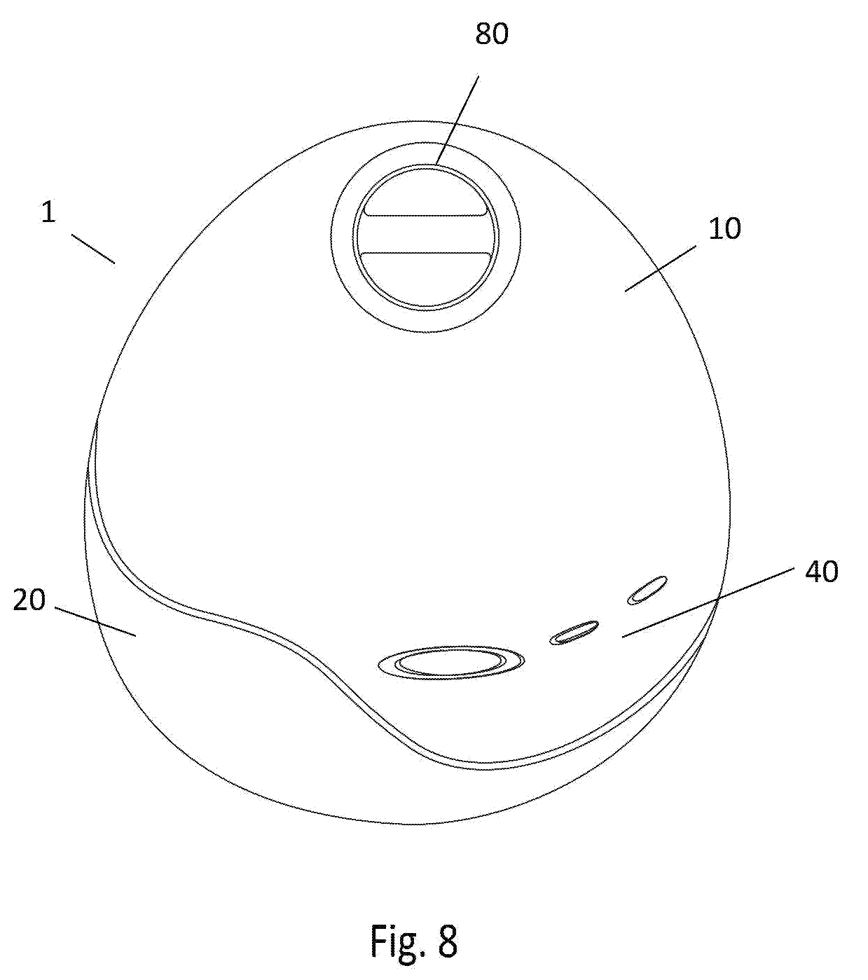

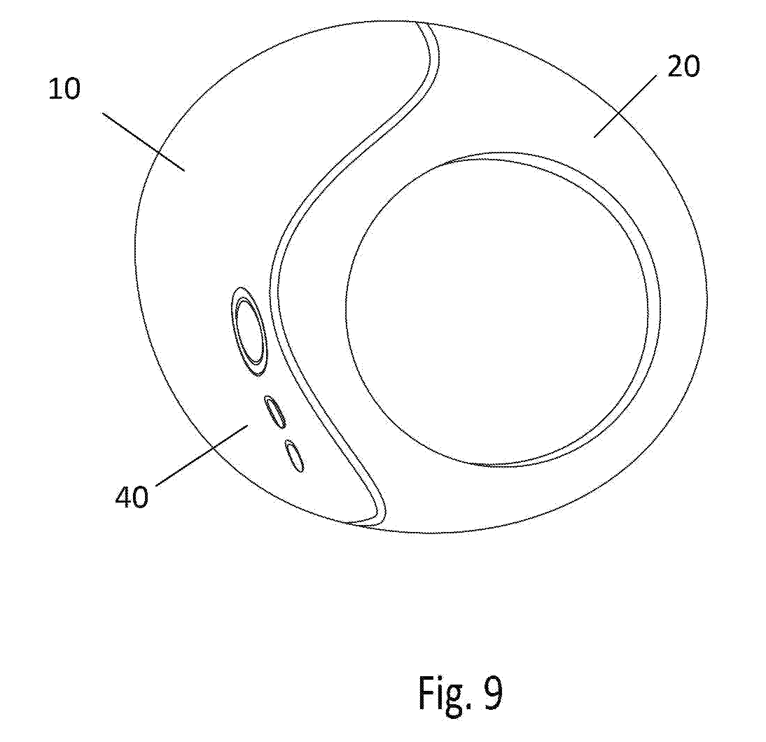

The current invention is directed to steam cleaning device 1 as shown in FIGS. 1-9, and in particular to one which is specifically designed to be used with baby products. The steam cleaning device utilizes a pressurized water vapor system that guarantees a 99.9% sanitation effectiveness every use.

The steaming device 1 is a oval shape in the preferred embodiment. The device 1 has a lid 10 and a base 20. The lid 10 is auto-locking to add additional protection. The lid 10 can be removed to place the baby products like bottoms 60 or nipples 70 in the device 1 to be cleaned. The lid 20, in the preferred embodiment, will be made of a clear, hard plastic. The lid 10 will auto locking for protection purposes.

The steaming device 1 has a base 20 with a flat bottom 100. The base 20 has a dual chamber heating steam system for maximum clean and dirty water filtration and separation. The clean water is converted into steam and the condensed water from the steam is held in a different chamber from the clean water.

The device 1 uses a vapor sterilization water pressurized vapor system which in the preferred embodiment must reach a optimum temperature of 171° F. no more than 190 degrees F. or it will bake on the food.

Located on the side of the base 20, the steam cleaner 1 has a set of digital timers 40 that control the heat. In the preferred embodiment, the digital time 40 illuminates green, yellow and red. Red means hot. Yellow equals cool down. Green means cool to the touch and ready for use.

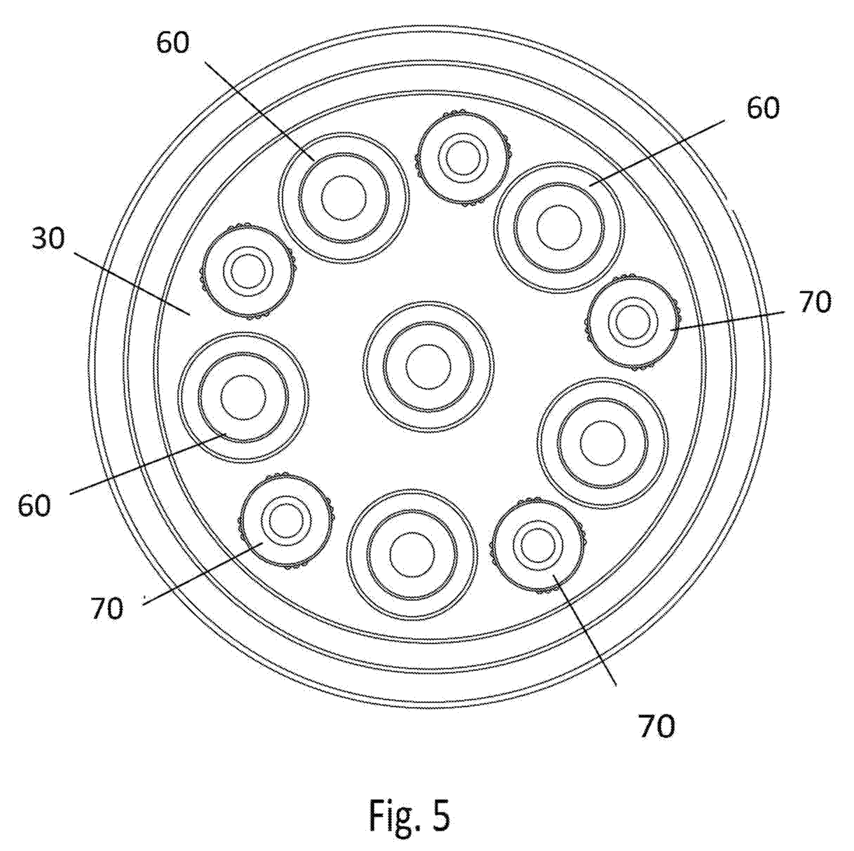

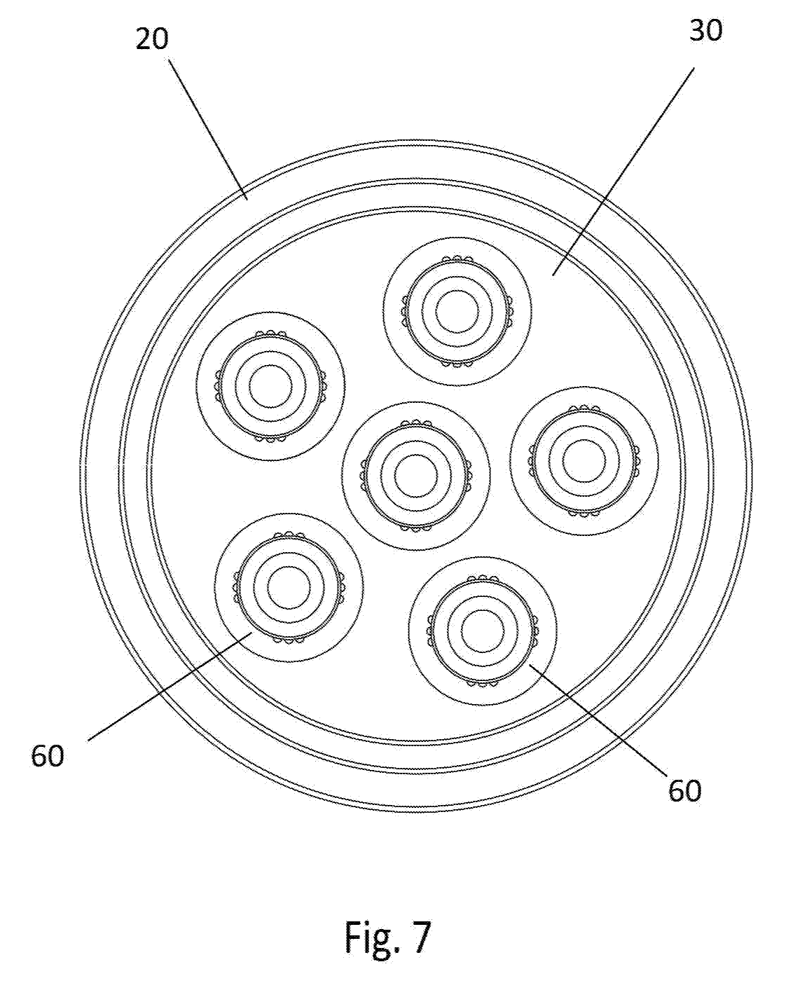

The base 20 will have a removable set of guides 30. The guides 30 are circular in shape and fit on to a ledge in the base 20. As shown in FIGS. 5, 6 and 7, the guides 30 have openings into which the baby products, like bottles 60, can be placed and for the steam to be let out from. The placement of the openings can be different from guide 30 to guide 30 depending on the baby products that the user wants to clean. The user can also heat up bottles of baby formula or breast milk as shown in FIGS. 1 and 7. In the preferred embodiment, the steam cleaner is designed to hold six standard size bottles 60.

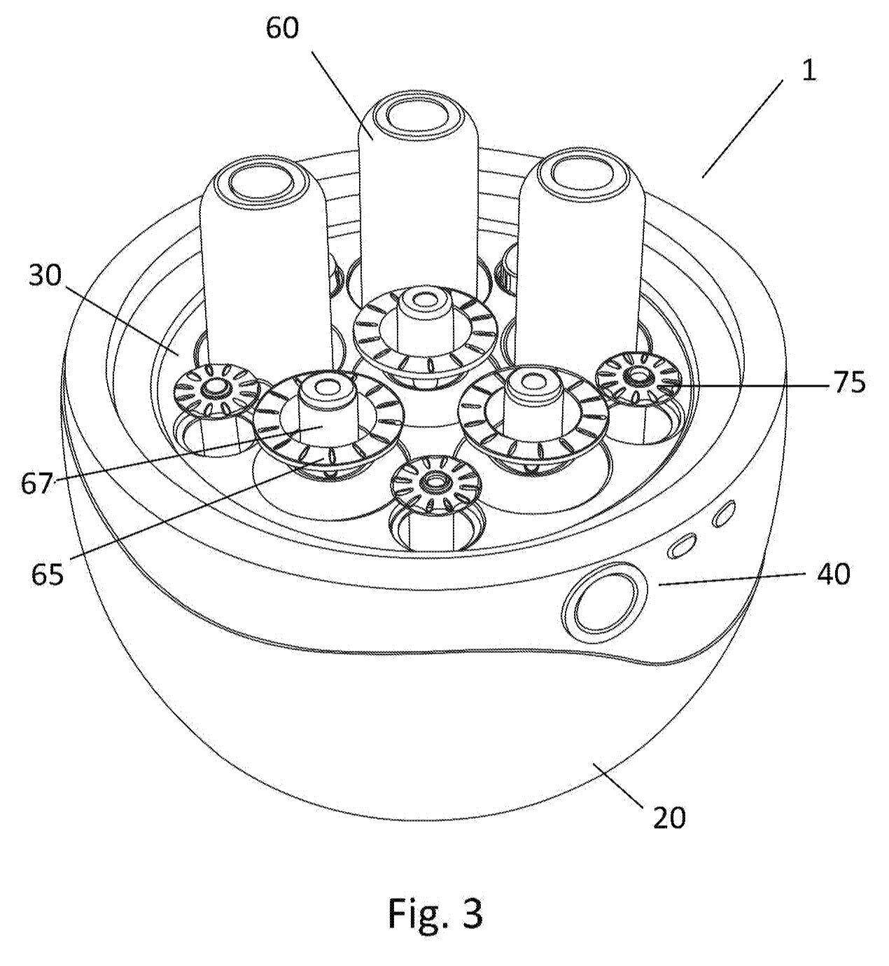

A guide 30, as shown in FIGS. 3, 4 and 6, can be used to hold and clean the nipples 70 as well as the bottles 60. In this configuration, the bottles 70 face down. They are placed on stabilizing rings 65 and bottom holders 67. The stabilizing rings 65 are ring structures on which the bottles 60 are placed. The rings 65 have ridges that are perpendicular to the center of the ring. The ridges hold the bottles up from the surface of the rings 65 allowing proper flow of the steam and the condensed water for a proper cleaning of the bottles. The bottom holders 67 are hollow tubes that go into the bottle to hold the bottle in place and to inject the steam through the tubes inside the bottle for a proper cleaning. There are nipple rings 75 onto which the nipples 70 are placed for cleaning. The nipple rings 75 also have the ridges that are perpendicular to the center for proper steam and condensed water to flow for a proper cleaning.

The steam cleaner is ergonomic in design with a sleek eye appealing space saving design.

The steam cleaner will have a heating element to heat water into steam and a power source such as an AC plug or batteries. The Batteries would water resistant to prevent corrosion also preventing short circuits and could be rechargeable. The steam cleaner could also use solar as an adaptive power supply in a travel situation the panel would be plugged into the same port as the AC cord.

### Advantages

The current invention is the only steam cleaner that utilizes a water vapor system that guarantees a 99.9% sanitation effectiveness every use. The steam cleaner 1 is designed to work with green solutions with no added chemicals.

While the methods disclosed herein have been described and shown with reference to particular steps performed in a particular order, it is understood that these steps may be combined, sub-divided, or reordered to form an equivalent method without departing from the teachings of the embodiments. Accordingly, unless specifically indicated herein, the order and grouping of the steps is not a limitation of the embodiments. Furthermore, methods and mechanisms of the embodiments will sometimes be described in singular form for clarity. However, some embodiments may include multiple iterations of a method or multiple variations of a mechanism unless noted otherwise. For example, when a connection is disclosed in one embodiment, the scope of the embodiment is intended to also cover the use of multiple connections.

Certain features of the embodiments, which may have been, for clarity, described in the context of separate embodiments, may also be provided in various combinations in a single embodiment. Conversely, various features of the embodiments, which may have been, for brevity, described in the context of a single embodiment, may also be provided separately or in any suitable sub-combination. Embodiments described in conjunction with specific examples are presented by way of example, and not limitation. Moreover, it is evident that many alternatives, modifications, and variations will be apparent to those skilled in the art. It is to be understood that other embodiments may be utilized and structural changes may be made without departing from the scope of the embodiments.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

That which is claimed is:

1. A device comprising:
a steam cleaner with a base and a top with exchangeable guides that fit into the base that hold baby products.

2. A device according to claim 1 comprising:
having a dual chamber steaming means.

3. A device according to claim 1 comprising:
having the steamer be oval in shape.

4. A device according to claim 1 comprising:
Where said base has a flat bottom.

5. A device according to claim 1 comprising:
Where said base has a dual chamber heating steam system.

6. A device according to claim 1 comprising:
Where said base has a dual chamber heating steam system.

7. A device according to claim 1 comprising:
Having a set of digital timers that control the heat.

8. A device according to claim 7 comprising:
Where the digital time illuminates green, yellow and red.

9. A device according to claim 8 comprising:
Where red means hot, yellow equals cool down and green is cool to the touch and ready for use.

10. A device according to claim 1 comprising:
Where the base has a ledge on the inside and the guides fit into said ledge.

11. A device according to claim 1 comprising:
Where the guides have openings into which the baby products can be placed.

12. A device according to claim 1 comprising:
Where the guides hold six standard size baby bottles.

13. A device according to claim 1 comprising:
Where the device heats up bottles of baby formula or breast milk

14. A device according to claim 1 comprising:
Where the guide has a plurality of stabilizing rings.

15. A device according to claim 14 comprising:
Where the stabilizing rings are ring structures on which bottles are placed.

16. A device according to claim 14 comprising:
Where the stabilizing rings have ridges that are perpendicular to the center of the ring.

17. A device according to claim 16 comprising:
Where the ridges hold the bottles up from the surface of the rings allowing proper flow of the steam and the condensed water for a proper cleaning of the bottles.

18. A device according to claim 1 comprising:
having hollow tubes that go into the bottle to hold bottles in place and to inject the steam through the tubes inside the bottle.

19. A device according to claim 1 comprising:
Where the guide has a plurality of nipple rings onto which nipples are placed for cleaning.

20. A device according to claim 19 comprising:
Where the nipple rings have the ridges that are perpendicular to the center for proper steam and condensed water to flow for a proper cleaning.
